(19) United States
(12) Patent Application Publication
(10) Pub. No.: US 2006/0084817 A1
(43) Pub. Date: Apr. 20, 2006

(54) Synthesis of aldonolactones, aldarolactones, and aldarodilactones using gas sparging

(76) Inventor: Henry Keith Chenault, Hockessin, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/173,463

(22) Filed: Jun. 30, 2005

Related U.S. Application Data
(60) Provisional application No. 60/584,835, filed on Jun. 30, 2004.

(51) Int. Cl. C07D 309/30

(52) U.S. Cl. 549/292

(57) ABSTRACT

Aldaric acids, aldonic acids, and their corresponding salts are cyclized to the corresponding lactone or dilactone using gas sparging to remove water.

### FIELD OF INVENTION

This invention is directed to processes for producing lactones or dilactones from aldonic acids, aldaric acids or aldarolactones, or salts thereof. The processes include dehydratively cyclizing a reaction mixture comprising a 5- to 8-carbon aldonic acid, 5- to 8-carbon aldaric acid or 5-to 8-carbon aldarolactone, or mixture thereof, in a solvent mixture, and removing water by gas sparging.

### BACKGROUND

Lactones and dilactones derived ultimately from renewable carbohydrate resources are highly functionalized monomers that are useful as synthetic intermediates, chiral starting materials, enzyme inhibitors, and monomers for polymer synthesis.

Aldaric acids and aldonic acids are oxidized derivatives of aldose carbohydrates. When only the aldehyde of an aldose is oxidized, an aldonic acid is formed. If both the aldehyde and terminal alcohol of an aldose are oxidized, an aldaric acid is formed. Lactones and dilactones can be produced from these acids via dehydrative cyclization, typically by heating the parent aldonic or aldaric acid under vacuum (Hirasaka, Y., Umemoto, K. Chem. Pharm. Bull. 1965, 13, 325-329). Recent publications and patents demonstrate that this technology has not changed for many years (U.S. Pat. No. 6,049,004). Even with heating under vacuum, conversion to the desired lactone is often incomplete (Conchie, J.; Hay, A. J.; Strachan, I.; Levvy, G. A. Biochem. J. 1967, 102, 929-941), requiring purification of the desired lactone by recrystallization (Isbell, H. S.; Frush, H. L. Bur. Standards J. Research 1933, 11, 649-664) or column chromatography. Furthermore, heating under vacuum often generates impurities due to thermal decomposition.

Hashimoto, et al. (Hashimoto, K.; et al., Makromol. Chem., Rapid Commun. 1990, 11, 393-396) disclose the synthesis of D-glucaro-1,4:6,3-dilactone by repeated lyophilization of glucaric acid from dioxane. Berends and Konings disclose the use of 1-butanol to effect the synthesis of aldonolactones by azeotropic removal of water (Berends, W.; Konings, J. Rec. Trav. Chim. 1955, 74, 1365-1370), but the method suffers from the formation butyl esters as by-products. While known processes may be acceptable for generating grams to tens of grams of material, they can be impractical for preparing tens to thousands of pounds of material. High vacuum, long residence time, and the high substrate surface area required by the solvent-free method are all impediments to practicing these methods on large scale.

What is needed, therefore, is a process that is feasible on a larger scale than previously reported methods and that will also generate low quantities of decomposition by-products.

### SUMMARY OF THE INVENTION

The present invention provides processes for preparing lactones or dilactones comprising the dehydrative cyclization of a reaction mixture comprising a 5- to 8-carbon aldonic acid, 5- to 8-carbon aldaric acid or 5-to 8-carbon aldarolactone, or mixture thereof, wherein the reaction mixture comprises a solvent mixture and about 10-90 weight % of solids, wherein the solvent mixture comprises about 5 to about 100 volume % of water, and wherein water is removed by gas sparging. The gas is preferably inert. Surprisingly, the processes, which rely on dehydrative cyclization, can be carried out in an aqueous medium and removal of water is effected by gas sparging.

One aspect of the present invention is a process for preparing a lactone or dilactone comprising:
a) providing a reaction mixture comprising:
   i) a solvent mixture comprising about 95 to about 0 volume % of a non-aqueous solvent and about 5 to about 100 volume % of water, based on the total volume of the solvent mixture; and
   ii) a starting material comprising one or more compounds selected from 5- to 8-carbon aldonic acids, 5-to 8-carbon aldaric acids, and 5- to 8-carbon aldarolactones;
b) optionally heating the reaction mixture;
c) removing by reduced pressure any non-aqueous solvent from the reaction mixture, to form a mixture substantially free of non-aqueous solvent;
d) optionally adjusting the mixture substantially free of non-aqueous solvent to a concentration of about 10 to about 90 weight % of solids; and
e) sparging the mixture substantially free of non-aqueous solvent, by passing a stream of an inert gas through the mixture substantially free of non-aqueous solvent, with heating and optional agitation.

In some embodiments, the reaction mixture comprises an equilibrium mixture of an aldaric acid and one or more of the corresponding aldarolactone or aldarodilactone, or an equilibrium mixture of an aldonic acid and the corresponding aldonolactone. In some embodiments the aldaric acid is glucaric acid. In some embodiments, the aldonic acid is gluconic acid.

In some embodiments, the aldonic acid, aldaric acid or aldarolactone contains one or more protected hydroxyl groups. The hydroxyl groups can be protected as ethers, acetals, carboxylic esters, or sulfonate esters.

In some embodiments, the 5- to 8-carbon aldonic acid, 5- to 8-carbon aldaric acid or 5- to 8-carbon aldarolactone is D, L, racemic or a nonracemic mixture in its enantiomeric configuration. The reaction mixture can also comprise an aldaric acid that has a plane of symmetry and thus exists in only a meso configuration.

In some embodiments, the aldonic acid, aldaric acid or aldarolactone is generated in situ from the corresponding Group I, Group II, or ammonium salt, or mixture thereof by acidification. The salt can be a sodium, potassium, lithium, cesium, magnesium, calcium, or ammonium salt, and the acid can be sulfuric acid, HCl, phosphoric acid, HF, oxalic acid, trifluoroacetic acid, or an acidic cation exchange resin. Optionally any precipitate formed during the generation of the aldonic acid, aldaric acid or aldarolactone in situ can be removed.

### DETAILED DESCRIPTION

The present invention provides processes for the preparation of a lactone or dilactone by dehydrative cyclization of a 5- to 8-carbon aldonic acid, 5- to 8-carbon aldaric acid or 5- to 8-carbon aldarolactone, or mixture thereof, in a reaction mixture, wherein the reaction mixture comprises a solvent mixture and about 10-90 weight % of solids, wherein the solvent mixture comprises about 5 to about 100 volume % water, and wherein water is removed by gas sparging.

The solvent mixture can comprise about 10 to about 90% of water and at least one non-aqueous solvent. The non-aqueous solvent can be removed by reduced pressure from the solvent mixture. The solvent mixture can comprise, for example, at least one of acetone, methyl ethyl ketone, methanol, ethanol, isopropanol and tetrahydrofuran. In some embodiments the solvent mixture comprises acetone and about 10 to about 50 volume % of water, based on the total volume of the solvent mixture.

The reaction mixture can comprise, for example, gluconic, mannonic, galactonic, idonic, allonic, altronic, gulonic, talonic, ribonic, xylonic, arabinonic, lyxonic, glucaric, mannaric, galactaric, idaric, allaric, altraric, ribaric, xylaric or arabinaric acid.

As used herein, an aldaric acid is a derivative of an aldose carbohydrate in which the terminal aldehyde and alcohol groups have been converted to carboxylic acids. An example of an aldaric acid is the aldaric acid derived from glucose, glucaric acid: HOOC—(CHOH)₄—COOH. Any aldaric acid that can form a lactone or dilactone is suitable for the instant invention, as described below. The aldaric acid can be in any enantiomeric form. Aldaric acid starting materials include but are not limited to glucaric (=gularic), mannaric, galactaric, idaric, allaric, altraric (=talaric), ribaric, xylaric, and arabinaric (=lyxaric) acids. Preferred are five to eight carbon aldaric acids; more preferred is glucaric acid; most preferred is D-glucaric acid.

Six-carbon aldaric acids that can form two cis-fused five-membered lactones (γ-lactones) do so and thus generate dilactone products. The other six-carbon aldaric acids and the five-carbon aldaric acids form monolactones as their ultimate lactonization products.

Pictured below are the ultimate products formed when six- and five-carbon aldaric acids are dehydratively lactonized. In cases where the starting material is optically active, only one enantiomeric product is pictured. One skilled in the art will recognize that the other enantiomeric starting material will form the enantiomeric product (e.g., L-mannaric acid gives L-mannaro-1,4:6,3-lactone) and that mixtures of stereoisomers, including racemates, form corresponding mixtures of stereoisomeric products. It is also understood that various salts of the aldaric acids can be converted into the free acid in situ and then lactonized.

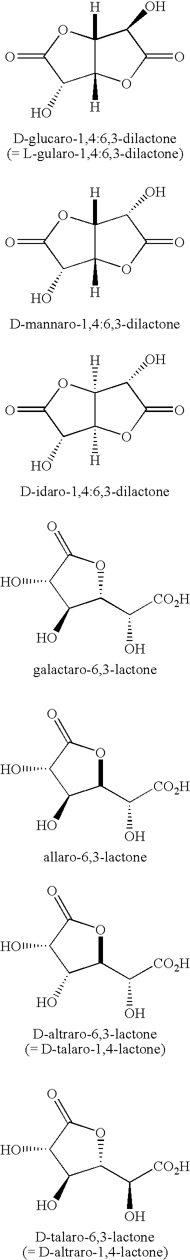

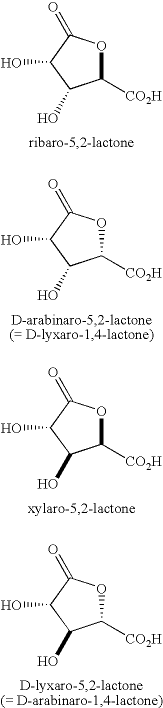

Because the molecules have carboxyl groups at both ends, there is potential for numbering from either end (e.g., D-glucaric acid has the same absolute structure as L-gularic acid, and D-altraro-6,3-lactone has the same absolute structure as D-talaro-1,4-lactone).

D-Glucaric acid (CAS Reg. No. 87-73-0, =L-gularic acid) gives D-glucaro-1,4:6,3-dilactone (CAS Reg. No. 826-91-5, =L-gularo-1,4:6,3-dilactone). L-Glucaric acid (CAS Reg. No. 5627-26-9, =D-gularic acid) gives L-glucaro-1,4:6,3-dilactone (=D-gularo-1,4:6,3-dilactone).

D-Mannaric acid (CAS Reg. No. 22076-54-60) gives D-mannaro-1,4:6,3-dilactone (CAS Reg. No. 2900-01-8). L-Mannaric acid gives L-mannaro-1,4:6,3-dilactone (CAS Reg. No. 214038-58-1, although this CAS registry number is incorrectly named L-mannonic acid di-γ-lactone).

D-Idaric acid (CAS Reg. No. 33012-63-4) gives D-idaro-1,4:6,3-dilactone. L-Idaric acid (CAS Reg. No. 80876-58-0) gives L-idaro-1,4:6,3-dilactone.

Galactaric acid (CAS Reg. No. 526-99-8, meso and thus optically inactive) gives (racemic) DL-galactaro-6,3-dilactone (=DL-galactaro-1,4-dilactone).

Allaric acid (CAS Reg. No. 527-00-4, meso and thus optically inactive) gives (racemic) DL-allaro-6,3-dilactone (=DL-allaro-1,4-dilactone).

D-Altraric acid (CAS Reg. No. 117468-78-7, =D-talaric acid) gives a mixture of D-altraro-1,4-lactone (CAS Reg. No. 91547-68-1, =D-talaro-6,3-lactone, although incorrectly named in CAS registry as D-talomucic acid 1,4-lactone) and D-altraro-6,3-lactone (CAS Reg. No. 91547-67-0, =D-talaro-1,4-lactone, although incorrectly named in CAS registry as D-talomucic acid 6,3-lactone). L-Altraric acid (CAS Reg. No. 117468-79-8, =L-talaric acid) gives a mixture of L-altraro-1,4-lactone (=L-talaro-6,3-lactone) and L-altraro-6,3-lactone (=L-talaro-1,4-lactone).

Ribaric acid (meso, CAS Reg. No. 33012-62-3) gives (racemic) DL-ribaro-5,2-lactone (CAS Reg. No. 85114-92-7, DL-ribaro-1,4-lactone).

D-Arabinaric acid (CAS Reg. No. 20869-04-9, =D-lyxaric acid) gives a mixture of D-arabinaro-1,4-lactone (=D-lyxaro-5,2-lactone) and D-arabinaro-5,2-lactone (=D-lyxaro-1,4-lactone). L-Arabinaric acid (CAS Reg. No. 608-54-8, =D-lyxaric acid) gives a mixture of L-arabinaro-1,4-lactone (=L-lyxaro-5,2-lactone) and L-arabinaro-5,2-lactone (=L-lyxaro-1,4-lactone).

Xylaric acid (meso, CAS Reg. No. 10158-64-2) gives (racemic) DL-xylaro-5,2-lactone (=DL-xylaro-1,4-lactone).

An aldonic acid, as used herein, is a derivative of an aldose carbohydrate in which the terminal aldehyde group has been converted to a carboxylic acid. An example of an aldonic acid is the aldonic acid derived from glucose, gluconic acid: HOOC—(CHOH)₄—CH₂OH. Any aldonic acid that can form a lactone is suitable for the instant invention, as described below. The aldonic acid can be in any enantiomeric form. Suitable aldonic acids include, but are not limited to, gluconic, mannonic, galactonic, idonic, allonic, altronic, gulonic, talonic, ribonic, xylonic, arabinonic, and lyxonic acids. Preferred are 5-8 carbon acids; most preferred is gluconic acid.

Pictured below are the 12 1,4-lactones (γ-lactones) formed by the 8 six-carbon and 4 five-carbon aldonic acids. Because aldonic acids have only one carboxyl group, they can form only one lactone ring. Some of the products shown below will be formed in the presence of their corresponding 1,5-lactone (δ-lactone), but the 1,4-lactone is usually the major product, especially at higher temperatures.

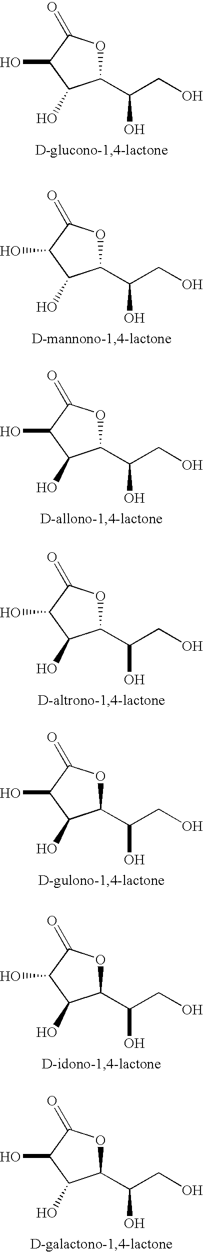

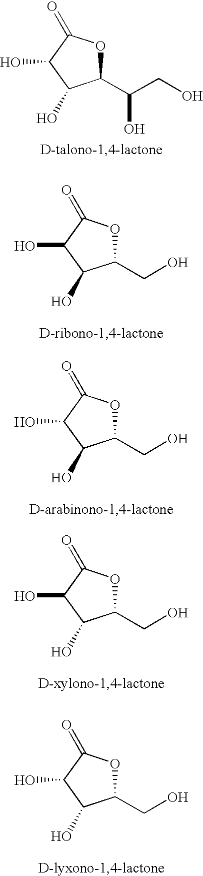

As with the aldarolactones above, only one enantiomeric form of each aldonolactone is pictured. One skilled in the art will recognize that the other enantiomeric starting material would give the enantiomeric product and that mixtures of stereoisomers, including racemates, would form corresponding mixtures of stereoisomeric products. It is also understood that various salts of the aldonic acids may be converted into the free acid in situ and then lactonized.

D-Gluconic acid (CAS Reg. No. 526-954) gives D-glucono-1,4-lactone (1198-69-2). L-Gluconic acid (CAS Reg. No. 157663-13-3) gives L-glucono-1,4-lactone (CAS Reg. No. 7446444-1).

D-Mannonic acid (CAS Reg. No. 642-99-9) gives D-mannono-1,4-lactone (CAS Reg. No. 26301-79-1). L-Mannonic acid (CAS Reg. No. 51547-37-6) gives L-mannono-1,4-lactone (CAS Reg. No. 22430-23-5).

D-Allonic acid (CAS Reg. No. 21675-42-3) gives D-allono-1,4-lactone (CAS Reg. No. 29474-78-0). L-Allonic acid gives L-allono-1,4-lactone (CAS Reg. No. 78184-43-7).

D-Altronic acid (CAS Reg. No. 22430-69-9) gives D-altrono-1,4-lactone (CAS Reg. No. 83602-36-2). L-Altronic acid gives L-altrono-1,4-lactone (CAS Reg. No. 119008-75-2).

D-Gulonic acid (CAS Reg. No. 20246-33-7, or CAS Reg. No. 66905-24-6 for the monohydrate) gives D-gulono-1,4-lactone (CAS Reg. No. 6322-07-2). L-Gulonic acid (CAS Reg. No. 526-97-6) gives L-gulono-1,4-lactone (CAS Reg. No. 1128-24-1).

D-Idonic acid (CAS Reg. No. 488-33-5) gives D-idono-1,4-lactone (CAS Reg. No. 161168-87-2). L-Idonic acid (CAS Reg. No. 1114-17-6) gives L-idono-1,4-lactone (CAS Reg. No. 1128-24-1).

D-Galactonic acid (CAS Reg. No. 576-36-3) gives D-galactono-1,4-lactone (CAS Reg. No. 2782-07-2). L-Galactonic acid (CAS Reg. No. 28278-17-3) gives L-galactono-1,4-lactone (CAS Reg. No. 1668-08-2).

D-Talonic acid (CAS Reg. No. 20246-35-9) gives D-talono-1,4-lactone (CAS Reg. No. 23666-11-7). L-Talonic acid gives L-talono-1,4-lactone (CAS Reg. No. 127997-10-8).

D-Ribonic acid (CAS Reg. No. 642-98-8) gives D-ribono-1,4-lactone (CAS Reg. No. 5336-08-3). L-Ribonic acid gives L-ribono-1,4-lactone (CAS Reg. No. 133908-85-7).

D-Arabinonic acid (CAS Reg. No. 488-30-2) gives D-arabinono-1,4-lactone (CAS Reg. No. 2782-09-4). L-Arabinonic acid (CAS Reg. No. 608-53-7) gives L-arabinono-1,4-lactone (CAS Reg. No. 51532-86-6).

D-Xylonic acid (CAS Reg. No. 526-91-0) gives D-Xylono-1,4-lactone (CAS Reg. No. 15384-37-9). L-Xylonic acid (CAS Reg. No. 4172-44-5) gives L-xylono-1,4-lactone (CAS Reg. No. 68035-75-6).

D-Lyxonic acid (CAS Reg. No. 526-92-1) gives D-lyxono-1,4-lactone (CAS Reg. No. 15384-34-6). L-Lyxonic acid (CAS Reg. No. 4172-43-4) gives L-lyxono-1,4-lactone (CAS Reg. No. 104196-15-8).

The starting materials can have one or more hydroxyl groups modified to give either a “deoxy” or a protected derivative. By “protected” is meant blocking the reactivity of a hydroxyl group with one or more reagents while a chemical reaction is carried out at an alternative reactive site of the same compound. Protecting groups are well known in the art and any suitable group can be used. Useful hydroxylprotecting groups include ethers, acetals, and carboxylic or sulfonate esters.

Since many aldonic and aldaric acids exist in solution in equilibrium with their lactone and (if possible) dilactone derivatives, the starting material may be a mixture of an aldonic or aldaric acid and its various lactone and (if possible) dilactone derivatives. Furthermore, since aldonic and aldaric acids generally exist in both D and L enantiomeric configurations, the starting material may be D, L, racemic (DL), or an unequal mixture of enantiomers. Some aldaric acids have a plane of symmetry and thus exist in only a meso configuration.

The starting aldonic or aldaric acid or corresponding lactone may be generated by acidifying a Group I, Group II, or ammonium salt of the parent acid or monolactone. Salts that may serve as precursors include but are not limited to sodium, potassium, lithium, cesium, magnesium, calcium, and ammonium salts. A mixture of salt forms having different cations may also be used as a precursor to the aldonic or aldaric acid. Acids useful for generating aldonic and aldaric acids by acidifying precursor salts include strong mineral acids, carboxylic acids, or polymer bound acids, such as but not limited to sulfuric, hydrochloric, phosphoric, hydrofluoric, oxalic, and trifluoroacetic acids, hydrogen chloride, hydrogen fluoride, and polymeric or solid-phase acids (e.g., strongly acidic cation exchange resins). The starting material may be generated as a solution in water, a suitable organic solvent such as acetone, or a mixture of said solvent and water. Any precipitate formed may optionally be removed by any means, such as filtration, before proceeding.

The starting material may optionally be a mixture of different aldonic and/or aldaric acids having different numbers of carbon atoms, different diastereomeric configurations, and/or different numbers of carboxylic acid groups. The mixtures can also be generated in whole or in part by acidifying the appropriate precursor salts.

In the instant processes, the starting material can be a mixture of one or more of an aldonic acid, an aldaric acid, an aldonolactone, an aldarolactone, and an aldarodilactone. The mixture can be an equilibrium mixture of an aldaric acid or an aldonic acid with its corresponding aldarolactone, aldonolactone, and/or its corresponding aldarodilactone if one exists. Preferably the starting materials contain 5-8 carbons.

The starting materials are dissolved in a suitable solvent that is comprised of about 5% to about 100 volume % of water to form the reaction mixture. By suitable it is meant any solvent or mixture of solvents that is substantially inert to all reagents and products, will dissolve the starting materials, and has a boiling point low enough to be removed at a temperature lower than the decomposition temperature of the reactants and products. Suitable solvents include but are not limited to water, ketones such as acetone and methyl ethyl ketone, alcohols such as methanol, ethanol, and isopropanol, and ethers such as THF (tetrahydrofuran). Preferred solvents include water, acetone, and mixtures thereof. Such mixtures can contain from 10 to 90% water by volume, based on the total volume of the solvent mixture, and in some preferred embodiments, from 10 to 50% of water.

If there is a solvent present other than water, the solvent can be removed by reduced pressure, such as distillation under reduced pressure. This can be performed at any temperature and pressure that is not detrimental to the reagents or products. Preferably, the solvent is one that boils at a lower temperature than water, facilitating its removal. If necessary, water is then added to the starting solution to form a mixture that is substantially free of non-aqueous solvents, which comprises about 10 to 90 weight % solids, preferably 50 to 80 weight % solids, based on the total weight of the mixture. A mixture “substantially free of non-aqueous solvents”, as used herein, means a mixture that contains little or no water, i.e., preferably about 0.5 weight percent or less of any non-aqueous solvents, or less. A mixture substantially free of non-aqueous solvents may be referred to herein in shorthand manner as an “aqueous mixture”. However, such usage, when used in reference to the mixture remaining after removal of non-aqueous solvent as described hereinabove, is not intended to require the complete absence of any non-aqueous solvent. On the other hand, aqueous mixtures that are free of non-aqueous solvents are also intended to be within the scope of the invention.

The concentrated aqueous solution is next sparged with a stream of gas. Any gas is suitable provided it does not react with or interact with the reactants, products, or solvent. Preferably the gas is dry air, dry nitrogen, dry argon; more preferably dry nitrogen. The gas used for sparging may be pre-heated to make the removal of water more efficient. Additionally, particularly if the volume of the solution is greater than 25 mL, stirring or other agitation may be used to improve the efficiency of water removal. The sparging removes water, both the water already present as solvent plus the water generated during the lactonization reaction in order to drive lactone (or dilactone) formation to completion.

The pressure during the cyclization procedure is preferably from about 1 to about 200 KPa. Stirring or agitation can optionally be used. By external heating or the use of pre-heated gas for sparging, the temperature of the mixture being dehydrated is kept at about 80 to about 130° C. The time required depends on the scale and geometry of the reactor but is typically from about 0.5 to about 5 hours. Completion can be determined by any method, such as periodically removing aliquots and analytically determining % conversion, or by monitoring refractive index or viscosity and color of product melt and absence of condensation. When the lactonization is complete, the stirring and sparging are discontinued, and the resulting syrup is allowed to cool to room temperature. The product can be purified using known methods, such as, for example, recrystallization.

It is highly desirable during the process that the solvent-free mixture of the starting materials remains liquid at only moderately high temperatures, for example 80-100° C., due to melting point depression. Even essentially pure lactone, for example glucaro-1,4:6,3-dilactone, remains a syrup during synthesis at temperatures below its melting point (132° C.) because its crystallization is usually slow kinetically. The sparging removes water in order to maintain this concentration. As illustration, when cyclizing gluconic acid to gluconolactone (a monolactone), two lactones are formed: 1,4- and 1,5-(or γ and δ). Melting point depression allows them to be formed as a melt at 118-124° C. when the melting points of the two pure lactones are 133-135° C. and 150-152° C., respectively.

### EXAMPLES

Materials and Methods used in the Examples

Calcium D-glucarate tetrahydrate (D-saccharic acid, calcium salt),

Spectrum Chemicals, 1001, FW 320.27

Sulfuric Acid, reagent grade, 95-98%, FW 98.07, d 1.84

Acetone, reagent grade, 99.5+%

D-Gluconic acid, 50 weight % in water, Sigma, G-1139, FW 196.2

### Example 1

Sulfuric acid (50.0 g, 0.500 mole) was added over a period of 30 minutes to a stirred suspension of calcium D-glucarate tetrahydrate (160.15 g, 0.500 mole) in 500 mL of 95:5 acetone-water (prepared by mixing 475 mL of acetone with 25 mL of water).

The stirred mixture was heated at reflux for 4 hours, allowed to cool to room temperature (20-25° C.), stirred at room temperature for 1-2 hours, and then filtered with suction to remove the precipitated calcium sulfate. At no time did the reaction become homogeneous. The precipitate was washed three times with 150 mL of 95:5 acetone-water, each time suspending the precipitate in the solvent and then sucking the solvent through.

Acetone was removed from the combined filtrate and washings by distilling under reduced pressure (pot temperature 30° C.). The concentrated aqueous solution was stirred mechanically with a stream of dry nitrogen passing through and over the surface of the solution. The solution was then heated to 120-130° C. for 2-3 hours, with continued stirring and nitrogen-sparging, to remove water.

Stirring and sparging was then discontinued, and the reaction mixture was allowed to cool to room temperature. The glassy product (85% yield, 92-94% pure) may be further purified by recrystallization. Analysis was performed by ¹H NMR and by GC (silylation with BSTFA-TMSCI, J&W DB-17MS 30 m×0.32 mm×0.25 m column, oven temperature 120-300° C.).

### Example 2

A 50 wt % solution of D-gluconic acid in water (7.6 g) was sparged with a stream of dry nitrogen. With continued sparging, the solution was heated to 112 to 118° C. for 1 hour and then 118 to 124° C. for 1 hour. Upon cooling to room temperature, the syrup solidified to a glassy solid that ¹H and ¹³C NMR indicated to be a 2:1 mixture of D-glucono-1,4-lactone and D-glucono-1,5-lactone.

What is claimed is:

1. A process for preparing a lactone or dilactone comprising:
a) providing a reaction mixture comprising:
i) a solvent mixture comprising about 95 to about 0 volume % of a non-aqueous solvent and about 5 to about 100 volume % of water, based on the total volume of the solvent mixture; and
ii) a starting material comprising one or more compounds selected from 5- to 8-carbon aldonic acids, 5- to 8-carbon aldaric acids, and 5- to 8-carbon aldarolactones;
b) optionally heating the reaction mixture;
c) removing by reduced pressure any non-aqueous solvent from the reaction mixture, to form a mixture substantially free of non-aqueous solvent;
d) optionally adjusting the mixture substantially free of non-aqueous solvent to a concentration of about 10 to about 90 weight % of solids; and
e) sparging the mixture substantially free of non-aqueous solvent, by passing a stream of an inert gas through the mixture substantially free of non-aqueous solvent, with heating and optional agitation.

2. The process of claim 1 wherein the gas used for sparging is dry air, dry nitrogen or dry argon.

3. The process of claim 1 wherein the solvent mixture comprises 10 to 90 volume percent of water and at least one non-aqueous solvent, and where the non-aqueous solvent is removed from the solvent mixture by reduced pressure before sparging.

4. The process of claim 3 wherein the solvent mixture comprises at least one selected from acetone, methyl ethyl ketone, methanol, ethanol, isopropanol and tetrahydrofuran.

5. The process of claim 3 wherein the solvent mixture comprises acetone and 10 to 90 volume percent water, based on the total volume of the solvent mixture.

6. The process of claim 1 wherein the reaction mixture comprises one or more acid selected from: gluconic, mannonic, galactonic, idonic, allonic, altronic, gulonic, talonic, ribonic, xylonic, arabinonic, lyxonic, glucaric, mannaric, galactaric, idaric, allaric, altraric, ribaric, xylaric and arabinaric acids.

7. The process of claim 1 wherein the reaction mixture comprises an equilibrium mixture of an aldaric acid and one or more of the corresponding aldarolactone or aldarodilactone, or an equilibrium mixture of an aldonic acid and the corresponding aldonolactone.

8. The process of claim 1 wherein the aldonic acid, aldaric acid or aldarolactone has one or more protected hydroxyl groups.

9. The process of claim 8 wherein the hydroxyl groups are protected as ethers, acetals, carboxylic esters or sulfonate esters.

10. The process of claim 1 wherein the aldaric acid is glucaric acid or where the aldonic acid is gluconic acid.

11. The process of claim 1 wherein the aldonic acid, aldaric acid or aldarolactone is D, L, racemic or a nonracemic mixture in its enantiomeric configuration.

12. The process according to claim 1 wherein the reaction mixture comprises an aldaric acid that has a plane of symmetry and thus exists in a meso configuration.

13. The process of claim 1 further comprising generating the aldonic acid, aldaric acid or aldarolactone in situ from a corresponding Group I, Group II, or ammonium salt thereof, or mixture of said salts.

14. The process of claim 13 wherein the salt is a sodium, potassium, lithium, cesium, magnesium, calcium, or ammonium salt.

15. The process of claim 14 wherein the salt is calcium glucarate.

16. The process of claim 13 wherein the aldonic acid, aldaric acid or aldarolactone is generated in situ via the addition of sulfuric acid, HCl, phosphoric acid, HF, oxalic acid, trifluoroacetic acid, or an acidic cation exchange resin.

17. The process of claim 13 further comprising removal of precipitate formed during the generation of the aldonic acid, aldaric acid or aldarolactone in situ.

18. The process of claim 1 wherein the solvent mixture comprises 10 to 50 volume percent of water and at least one non-aqueous solvent selected from: acetone, methyl ethyl ketone, methanol, ethanol, isopropanol and tetrahydrofuran.

19. The process of claim 1 wherein the gas is pre-heated prior to sparging.

20. The process of claim 1 wherein the process is carried out at a pressure of 1 to 200 kPa.
